(19) United States
(12) Patent Application Publication
(10) Pub. No.: US 2022/0061918 A1
(43) Pub. Date: Mar. 3, 2022

(54) LASER SOURCE, LASER DEVICE AND METHOD OF CUTTING A TISSUE

(71) Applicant: ADVANCED OSTEOTOMY TOOLS - AOT AG, Basel (CH)

(72) Inventors: Alfredo E. BRUNO, Biel-Benken (CH); Klaus Olaf BÖRNSEN, Denzlingen (DE); Michael PEYER, Bern (CH); Reinhard KELNBERGER, Krailling (DE)

(21) Appl. No.: 17/414,389

(22) PCT Filed: Dec. 20, 2019
(86) PCT No.: PCT/EP2019/086514

§ 371 Date: Jun. 16, 2021

(30) Foreign Application Priority Data
Dec. 21, 2018 (CH) 01596/18

(51) Int. Cl. A61B 18/20

(52) U.S. Cl. CPC A61B 18/201; A61B 2018/00577

(57) ABSTRACT

A laser source (101) comprises: (i) a first beam generating configuration (111, 112, 113) adapted to generate a pulsed primary ablating laser beam (162) with pulses having a first emission spectrum and a first temporal pulse width to ablate one type of tissue, (ii) a second beam generating configuration (121, 122, 123) adapted to generate a pulsed secondary ablating laser beam (163) with pulses having a second emission spectrum different from the first emission spectrum and a second temporal pulse width to ablate another type of tissue different than the one type of tissue ablated by the primary laser beam (162), (iii) a third beam generating configuration (121, 122, 123, 126) adapted to generate a pulsed analysis laser beam (161) with at least one pulse having a third emission spectrum and a third temporal pulse width shorter than the first temporal pulse width and shorter than the second temporal pulse width, and (iv) a beam directing optics (125) with beam aligning elements adapted to align the primary ablating laser beam, the secondary ablating laser beam (163) and the analysis laser beam (161) such that the laser source (101) propagates the laser beams (160) along a same propagation path.

### TECHNICAL FIELD

The present invention relates to a laser source according to the preamble of independent claim 1 and more particularly to a laser device having such laser source and a method of cutting a tissue. Such laser sources configured to propagate plural laser beams can be useful in many applications or fields.

### BACKGROUND ART

For cutting and drilling materials in various technical fields it has become increasingly popular to use apparatuses which apply a laser beam to the material. Today, in industrial applications such cutting or drilling is widespread since it allows for efficiently and flexibly process work pieces at high precision. Also, for cutting human or animal hard tissue such as bones, cartilages or the like cutting and drilling with laser is more and more applied. For example, in computer assisted surgery it is known to use laser beams as cutting instruments. More particularly, e.g., in WO 2011/035792 A1 a computer assisted and robot guided laser osteotomic medical device is described which allows for precise and gentle drilling and cutting of bone and other human or animal hard and also soft tissue.

More specifically, laser tissue ablation of soft biological tissue is being used in dermatology, urology, oncology, neurosurgery and other fields, where cutting of tissue and blood coagulation is important. For such purpose different laser systems like Thulium (Tm), Holmium (Ho), Neodymium (Nd) or Erbium (Er) embedded in various solid state glasses or crystals machined in the form of rods lasing in the infrared (IR) part of the spectrum which are pumped by either flash lamps (FLs) or laser diodes (LDs) are commonly used. Also, LDs are used for the purpose of stopping bleeding. Nd:YAG lasers are used for soft tissue cutting in urology, dentistry and other oral surgery areas. Low intensity Nd:YAG lasers are use against retina detachment and in other eye surgeries too. Another major area for Nd:YAG lasers are lipolysis providing against mechanical liposuction faster healing, less bleeding and less advert events and better results. Also, these lasers are used in a number of dermatology applications and in plastic surgery applications. Furthermore, CO₂ lasers have also been used in these fields in the past.

A situation often encountered when cutting or ablating tissue, e.g. in surgery, is that the type of the target tissue changes with increasing cutting depth or along the cut. Such change of tissue type in inhomogeneous tissues may decrease efficiency of the laser ablation or in some cases even stop the cutting or ablation process. For example, when cutting a bone, such as cutting a complete femur transversally, the tissue changes from the outer hard part being a cortical and spongious bone to a central part, i.e. the medulla, widely consisting of fatty tissue. These two types of tissue are more efficiently ablated by laser beams of two different distinct wavelengths. This, primarily because the bone tissue contains sufficient water to be ablated with a given laser beam while the inner fatty tissue has no or negligible water content, but it will be better ablated with a different laser beam. Even when using a water solution spray to cool the surface that is known to enhance the ablation efficiency, the fatty tissue is hydrophobic and the cutting efficiency when using a beam wavelength from, e.g., an Er:YAG laser emission line around 3 μm that is strongly absorbed by water, the ablation process remains inefficient.

Therefore, there is a need for a device, system or method allowing to efficiently cutting an inhomogeneous target tissue.

### DISCLOSURE OF THE INVENTION

According to the invention this need is settled by a laser source as it is defined by the features of independent claim 1, by a laser device as it is defined by the features of independent claim 15, and by a method as it is defined by the features of independent claim 25. Preferred embodiments are subject of the dependent claims.

In one aspect, the invention is a laser source comprising: (i) a first beam generating configuration adapted to generate a pulsed primary ablating laser beam with pulses having a first emission spectrum and a first temporal pulse width; (ii) a second beam generating configuration adapted to generate a pulsed secondary ablating laser beam with pulses having a second emission spectrum different from the first emission spectrum and a second temporal pulse width; (iii) a third beam generating configuration adapted to generate a pulsed analysis laser beam with at least one pulse having a third emission spectrum, which can be the same as the first or second emission spectrum, and a third temporal pulse width shorter than the first temporal pulse width and shorter than the second temporal pulse width; and (iv) a beam directing optics with beam aligning elements adapted to align the primary ablating laser beam, the secondary ablating laser beam and the analysis laser beam such that the laser source propagates the laser beams along a same propagation path.

The target tissue can particularly be a human or animal natural hard or soft tissue. Specifically, the target tissue can be a bone tissue or bone such as the femur.

The term “laser” can generally relate to a device or arrangement which is configured to generate a laser beam, or which emits light through a process of optical amplification based on the stimulated emission of electromagnetic radiation. Laser is an acronym for “light amplification by stimulated emission of radiation”. Laser may differ from other sources of light in that it emits light coherently. Such spatial coherence can allow a laser to be focused to a tight spot, which makes applications such as cutting or lithography possible. The beam generating configurations may in some instances be referred to as lasers.

The term “pulse” or “laser pulse” can relate to a comparably short-time laser beam preferably of a given wavelength having a specific temporal width, shape and power. In connection with generating laser pulses the term “firing” is used herein, which refers to the activation of one of the beam generating configurations or lasers of the laser source such that a pulse of electric voltage of a given voltage, current and temporal profile results.

In general, tissue ablation by pulsed laser beams occurs by various physical effects. In the most common cases, the laser light is absorbed by molecules like proteins, lipids, collagens and or other biological compounds. The conversion of absorbed laser energy leads to thermal heat resulting in a strong and rapid temperature increase. During this process, most often, molecules in the tissue are directly degraded and converted into debris being ejected from the ablation spot. This can be referred to as ablation by a direct ablation process of thermal nature. Such process can undesirably lead to carbonization of the tissue precluding subsequent healing. Therefore, the conditions for ablation have to be precisely optimized and controlled during the cutting process.

In addition to this direct ablation, there is also an indirect ablation process when using a water spray to cool and humidify the region of the tissue being ablated. Water droplets and/or a water film condensed at the surface of the tissue being ablated by a laser beam can be fragmented with plenty of kinetic energy provided by the laser beam pulses. These fragments may collide with walls of the tissue and thus ablating it. Such process can in certain conditions be even the main or sole contributor of the ablation as, e.g., in the case of tooth tissue, i.e. in dentistry applications. An example in bone surgery can be the indirect or cold ablation of hard tissues like bone and collagen tissue, or the indirect ablation of hydrophobic tissues such as the fatty central part of the femur, i.e. the medulla of the femur.

In connection with the invention, it has been observed that, for cutting tissues and particularly biological tissues with laser beams, it is not as important to make a distinction between soft and hard tissue as extrapolated when using mechanical tools but it is more appropriate to distinguish between hydrophilic or water containing tissue such as cells with water and hydrophobic tissues which are also associated with low water amount such as cells of nerves and fatty tissues. Furthermore, because of the hydrophobic character of these tissues, water cannot adhere or condense on the surface being ablated implying that the indirect ablation process mentioned above does not efficiently apply. Rather, in such cases a direct ablation process is beneficial since a proper wavelength can be selected to be efficiently absorbed by the hydrophobic tissue in question. Indeed, it is observed that on a hydrophobic surface, such as of a medulla in a femur, a water film from water droplets from an aqueous spray withdraws itself quickly from the surface leaving the surface dry such that no or only little indirect ablation occurs. More specifically, to overcome laser ablation problems of hydrophobic tissues laser beams having other wavelengths than the ones used for hydrophilic tissues render better results. For example, the use of Nd:YAG lasers is suitable for the ablation of lipids and most biological hydrophobic materials. The ablation in these cases is mainly based on the direct absorption of laser energy as above defined, i.e. it is a direct ablation process. On the other hand, fat and lipids are in general chemically more stable resisting higher temperatures. However, fat and lipids are mainly melting and can be desorbed under the influence of a laser pulse. The degradation of fat and lipids, or their carbonization, is here a very minor process.

As mentioned, an important issue when cutting a tissue is to know a priori what type of tissue is to be encountered by the ablation laser to decide which of the type of ablation laser beam wavelengths will be used for the subsequent laser pulse. For this purpose, the laser source according to the invention provides the sending of one or more sampling pulses, i.e. one or more pulses of the analysis laser beam, e.g. to induce a high temperature plasma, and to ablate a small amount of the targeted tissue in the form of debris to be analysed by any suitable analytical method. Once the analytical method determines that the tissue in question has a sufficient amount of water to induce efficient ablation such as more than 1% of water, the laser source can be activated to emit either one of the primary ablation laser beam, or the secondary ablation laser beam, whichever is more suitable for the identified tissue. Vice versa, if the analysed tissue is found not to contain sufficient water for respective direct ablation and/or if its surface is hydrophobic, the laser source can be activated to emit the other one of the of the primary ablation laser beam or the secondary ablation laser beam. In particular, the laser source can be operated such that continuously or regularly the analysis laser beam is provided and the appropriate primary ablation laser beam or the secondary ablation laser beam is emitted in correspondence with the identified tissue type. Such process can be continued through the whole cutting or ablating process. For example, considering that many laser pulses are required to ablate, e.g., the cortical part of the femur before encountering the medulla, the firing of the analysing laser beam not necessarily needs to be performed after each pulse of the primary or secondary lasers beam. Rather, it can be sufficient to lase with the analysing laser beam every five or more pulses of any of the primary and secondary ablating laser beams until the medulla is encountered. Indeed, having a laser source providing two or more types of laser beams of different wavelengths sharing the same coaxial propagation path, allows a number of different modes of operation offering high flexibility in cutting the different biological tissues.

The term “coaxial” as used in connection with propagation path relates to a spatial relation between the propagating axes of different light beams. It has no meaning regarding temporal relations which may arise by having multiple pulsed laser beams. Furthermore, coaxial can also cover particularly comparable close parallel directions.

In accordance with the invention, the laser source generating coaxial or same propagation path primary and secondary laser beams with pulses having distinct emission spectra and distinct temporal pulse widths allows for providing two or more different modes of ablation depending on the targeted tissue. In particular, the laser source allows to switch from the primary ablation laser beam, such as a laser beam of an Er:YAG laser beam generating configuration in free-running mode, to the secondary laser beam such as a laser beam of a Nd:YAG laser beam generating configuration in free-running mode. The acronym “Er” represents Erbium, the acronym “Nd” represents Neodymium and the acronym “YAG” represents Yttrium Aluminium Garnet (Y₃Al₅O₁₂). A laser operating in free-running mode can refer to the laser emission when the resonator does not have a pulse-shortening device but approximately mimics the temporal profile of the pumping source (e.g. similar to the time width of the flash lamp or of a laser diode). Moreover, this switching can be based of the tissue type identified by means of sample tissue ablated by the analysis laser beam. For example, when cutting on tumour regions it might be advantageous to have higher spot temperatures to avoid spreading of active tumour material. Or, the spreading of infectious material from regions of infections. Additionally, the Nd:YAG laser beams may support the coagulation of blood and helps to keep the surgery field and path clear.

The laser source according to the invention can be used in many laser firing sequences or modes for the at least three laser beams. For example, the analysing laser beam can be fired constantly at the same frequency as the primary and/or secondary ablating laser beams laser beams, eventually, with a small shift so that they do not overlap and that there is sufficient time for the analytical system to identify the tissue to be ablated. However, this laser firing mode of operation could delay the whole process. In a case such as cutting transversally a femur, the firing sequence could be arranged in such a way that once that the analysis laser beam and the analytical system identify the outer cortical part of the femur then a given number of laser pulses, e.g. 10 laser pulses, is fired from either ablating laser beams before the next pulse from the analysis laser beam is fired again. In this case, the frequency or repetition rate of the analysis laser beam can be 1/10^{th} of the frequency of the primary or secondary ablating laser beams. Furthermore, it might also be possible to fire more than one pulse of the analysis laser beam to ensure that the identification of the tissue being ablated is with a high degree of precision. This example, e.g. for the case of cutting transversally a femur, can be independent of the firing frequencies of ablating laser beams that is the right laser for cortical bone continues until the medulla is encountered. Moreover, the laser source can be operated in such a way that the time between two subsequent pulses of any of the three laser beams not necessarily needs to be constant. For instance, if the analytical method used to analyse the debris generated by the analysis laser beam requires some time to be analysed, e.g. 1/10^{th} of a second, then the primary or secondary ablating laser beams should wait, e.g. for a trigger signal, to fire either laser beam depending on the type of tissue identified.

Thus, the laser source according to the invention allows for efficiently cutting an inhomogeneous target tissue such as a bone having different types of tissue. More specifically, bones having two types of tissues such as the femur, where one tissue is hydrophilic, i.e. having a considerable amount of water for direct ablation, and the other tissue is hydrophobic, such as the water content from a spray does not adhere to the surface of the cut or hole, and/or have a negligible small amount of water it is highly beneficial to use pulsed laser ablating beams with different wavelengths. With the here proposed laser source, cutting the femur or other similar tissue can be easier by using, e.g., the primary ablation laser beam for the cortical part and the secondary ablation laser beam for the medulla.

Preferably, the first beam generating configuration has a gain medium to generate the primary ablating laser beam, and the second beam generating configuration has a second gain medium different from the first gain medium to generate the secondary ablating laser beam. When setting up the laser source, the gain media can be chosen to the intended application of the laser source. In particular, appropriate gain media can be embodied in order to allow generation of ablating laser beams suitable to cut or ablate the types of tissue involved.

The third beam generating configuration can have an own gain medium to generate the analysis laser beam. This third gain medium can be the same as or different from any of the first and second gain media. However, preferably the third beam generating configuration preferably comprises the second gain medium. Like this the third gain medium can be used to generate the secondary ablating laser beam as well as the analysis laser beam. In such embodiment, also the third emission spectrum advantageously is the same as the second emission spectrum. This allows for a particularly efficient implementation of the laser source.

Preferably, the third beam generating configuration comprises a giant pulse former. In this context, the term “giant pulse former” relates to the formation of laser beam pulses with comparably high peak power such as, e.g., gigawatt peak power. It can also be referred to as pulse compressor since in advantageous embodiments the giant pulse is formed by compressing a pulse. Such giant pulse former allows for shaping or generating laser beam pulses which are particularly suitable for the analysis of the debris resulting from the analysis laser beam hitting the target tissue. In particular, it allows for providing a comparably short but high energy laser pulse which non-selectively ablates all types of tissue but to a comparably small amount only.

In one preferred embodiment, the giant pulse former has an opto-electronic element such as an active Q-switching device. Such optoelectronic element or active Q-switching device allows for efficiently providing sophisticated giant laser pulses which are particularly suitable for the involved target tissue such as biological tissue.

In another preferred embodiment, the third beam generating configuration comprises two resonator mirrors and the giant pulse former has an electro-mechanical rotator to which one of the two resonator mirrors of the third beam generating configuration is mounted. By rotating on of the resonator mirrors a giant pulse can be generated by comparably simple means when the two resonator mirrors are properly aligned during a short period of time.

In another embodiment a passive Q-switching element or device can be used. For the passive Q-switching a saturable absorber can be used which comprises of a material transmission of which increases when the intensity of light exceeds a threshold. The material may be an ion-doped crystal, a bleachable dye or a passive semiconductor. Initially, the loss of the absorber is high, but still low enough to permit some lasing action. A large amount of energy is in this way stored in the gain medium. As the power increases, the laser beam saturates the absorber, which rapidly reduces the resonator loss. This may bring the absorber into a state with low losses to allow efficient extraction of the stored energy. A very short pulse with high peak power, a so-called giant pulse, is thus generated. After the pulse, the absorber recovers to its high loss state.

The term “Q-switching mode” or Q-switching” as used herein can relate to an intra-cavity opto-electrical mechanical process or to a process carried out by means of a saturable absorber for gating the laser light to generate short laser pulses of light. Here active and passive Q-switching devices are suitable. Q-Switching can be achieved by putting a variable attenuator inside the laser's optical resonator. When the attenuator is functioning, light which leaves the gain medium does not return, and lasing cannot occur. This attenuation inside the cavity corresponds to a decrease in the Q factor, or quality factor, of the optical resonator. The variable attenuator is thus commonly called a “Q-switch”, when used for this purpose. A high Q factor corresponds to low resonator losses per roundtrip, and vice versa. Initially the laser medium may be pumped while the Q-switch is set to prevent feedback of light into the gain medium producing an optical resonator with low Q factor. This produces a population inversion, but laser operation cannot yet occur since there is no feedback from the resonator. Since the rate of stimulated emission is dependent on the amount of light entering the medium, the amount of energy stored in the gain medium may increase as the medium is pumped. Due to losses from spontaneous emission and other processes, the stored energy can reach some maximum level after a certain time. The medium can be said to be gain saturated. At this point, the Q-switch device is quickly changed from low to high Q, allowing feedback and the process of optical amplification by stimulated emission to begin. Because of the large amount of energy already stored in the gain medium, the intensity of light in the laser resonator can build up very quickly. This also may cause the energy stored in the medium to be depleted almost as quickly. The net result can be a short pulse of light output from the laser which may have a high peak intensity.

Preferably, the first emission spectrum has a maximum in a range of about 2'900 nm to about 3'000 nm, in a range of about 2'950 nm to about 2'980 nm or in a range of about 2'960 nm to about 2'970 nm, or of about 2'964 nm. Such an emission spectrum is particularly efficient to ablate hydrophilic tissue. The term “hydrophilic” as used herein can relate to a type of tissue having a considerable high amount of water so that direct ablation can be achieved. For example, such emission spectrum can be particularly suitable for cutting or ablating the cortical part of a bone such as the femur. Such emission spectrum can, e.g., be generated by an Er:YAG beam generating configuration.

Preferably, the second emission spectrum has a maximum in a range of about 1'000 nm to about 1'100 nm, in a range of about 1'050 nm to about 1'080 nm or in a range of about 1'060 nm to about 1'070 nm, or of about 1'064 nm. The term “hydrophobic” as used herein can relate to a type of tissue to which the water from a spray does essentially not adhere to the surface and/or which has a negligible amount of water such as less than about 1%. Such an emission spectrum can, e.g., be generated by an Nd:YAG beam generating configuration which can be particularly efficient to ablate hydrophobic tissue such as the medulla of a bone, e.g., the femur. One advantage of the Nd:YAG beam generating configuration can be that it is operable in two modes. In a free-running operation it produces long pulses, e.g. from about 100 μs to about 400 μs, and high power, e.g. about 100 mJ to about 1 J, which is the ideal laser for the secondary ablation laser beam in the UTL device.

The fundamental lasing spectrum of the Nd:YAG laser or primary ablating laser beam can be 1'064 nm which falls in the very convenient infrared (IR) spectral region suitable to ablate hydrophobic tissues. The same laser or laser spectrum can be used for the analysis (i.e. the analysis laser beam) in either its fundamental lasing spectrum of 1'064 nm. Alternatively, the third emission spectrum preferably has a maximum in a range of about 500 nm to about 560 nm, or in a range of about 520 nm to about 540 nm, or of about 532 nm. In particular, the third emission spectrum in its second-harmonic version (SHG) can be in the visible part of the spectrum of 532 nm beam. Such SHG can be generated under special conditions using frequency doubling crystals such as KDP crystals (Potassium dihydrogen-phosphate) or others. Because the SHG effect is a non-linear process in terms of pulse peak power, such visible frequency can be more efficiently obtained when the laser pulses are relatively short (e.g. less than 20 ns) as those obtained when the Nd:YAG laser is operated with a Q-switching device. The temporal profile of the 532 nm beam can be similar or slightly shorter than the temporal profile of the 1'064 nm beam, but its intensity can much lower such as a fraction of the fundamental intensity. Frequency doubling crystals are usually placed extra-cavity and after the intracavity Q-switching device and are to be adjusted at a particular angle with respect to the incoming 1'063 nm beam. Both laser beams may emerge for the frequency doubling crystal in a coaxially therefore, to use one or another laser beam it is necessary to use filters to block one of the two wavelengths or dichroic mirrors or prisms to separate the two colours.

Furthermore, the same gain medium could be operated with an intra-cavity optical (i.e. inside the optical resonator) or opto-electronic element, such as a Q-switching device, that shortens the pulses to, e.g., about 10 ns to about 20 ns in low power pulses, e.g. μJ, and thus ideal for the analysis laser beam. Or, if the pulses have longer time widths the energy per pulses should also be higher to be able to sustain ablation of the tissue and ionization and/or electronic excitation of the fragments in the debris; important can be that the peak power defined as the pulse energy/time width remains high as exemplified in the subsequent paragraph. An additional advantage of using an Nd:YAG laser is that tissue bleeding can be reduced if needed without the classical tissue carbonization effect. Also, a set of multiple laser pulses, such as about 2 to about 6 within a short and exact time frame, such as ns to μs, before the ablation lasers beams can reduce a shockwave, and/or can prepare the surface, and can increase the cutting speed.

A parameter of potential relevance in laser ablation as well as in the ionization of the fragments in the debris is not only the energy per pulse but the time width of the pulse that determines the peak power. For the typical cases mentioned above of, e.g., a pulse of the free running Er:YAG or Nd:YAG lasers having, e.g., 1 J of energy spread in 200 μs the peak power amounts to 5 kW whereas for the Q-switched Nd:YAG laser having, e.g., 100 mJ spread in 15 ns the peak power is 6.7 MW which is thousand times higher than when the same laser is run in free running mode. It can also be important to compare these values with those of a cw (continuous wave) laser operated at 10 W with very low peak power of also 10 W explaining the fact that cw lasers are not suitable for laser ablation.

For surgical applications involving the cutting of bones, it can be highly beneficial to use pulsed ablating laser beams with the emission spectra mentioned hereinbefore. Therefore, a laser source propagating ablation laser beams with the above emission spectra, allows for easy cutting of bones such as a femur by using the primary ablating laser beam for the cortical part and the secondary ablating laser beam for the medulla.

Preferably, the beam directing optics comprises a beam combining element arranged to combine the primary ablating laser beam, the secondary ablating laser beam and the analysis laser beam. In general, there are three possible ways to coaxially combine beams with difference wavelengths. The first way involves combining via a dichroic mirror. This mirror may reflect the laser beam with the higher wavelength and transmit the laser beam with the lower wavelength. The second way involves an opto-mechanical device, where, e.g., one or two adjustable mirrors are mounted on a electro-mechanical slide. The slide can have two positions, where each beam can be selected. The third way involves combining of the beams with mirrors mounted on a rotary axis such as galvanometer devices. In any way there are some opto-mechanical elements required to match the different divergence of the different laser sources, respectively to collimate each individual laser beam, and to deflect the beams individually be different mirrors for proper parallel alignment of the laser beams. In some embodiments, it can be favorable to have the analyzing laser beam in transmission for the beam mixing structure. When the beams are combined coaxially or parallel and considering that in most cases the beams are made up of pulses, the different pulsed beams usually are not propagating in the same space at the same time. In this sense the concept of coaxial or parallel can refer to two pulsed beams propagating through the same space but in slightly different time-periods.

Preferably, the first temporal pulse width and the second temporal pulse width are in a range of about 1 μs to about 1 ms or in a range of about 150 μs to about 300 μs. Such comparably long laser pulses can be particularly appropriate for ablating tissue to cut or drill the target tissue.

Preferably, the third temporal pulse width is in a range of about 1 ps to about 100 ns or in a range of about 1 ns to about 50 ns. Such comparably short laser pulses can be particularly suitable to ablate a short fraction of tissue at high temperature so that at the same time ions such as Ca⁺⁺, Na⁺, K⁺, as well as other ions, molecules or tissue fragments, are electronically excited to be detected easily by, e.g. laser-induced breakdown spectroscopy (LIBS), without harming it for analysis purposes.

Preferably, the laser source comprises at least one flash lamp as light source for the first beam generating configuration, the second beam generating configuration and/or of the third beam generating configuration. Thereby, each beam generating configuration can be equipped with an own flash lamp. Or, more efficiently, a flash lamp can be used to combine either beam generating configuration. In particular, one flash lamp can be provided as light source for the first beam generating configuration and another one flash lamp can be provided as light source for the second beam generating configuration and the third beam generating configuration together.

Such flash lamps allow for providing light pulses to the gain medium such that laser pulses are emitted which are particularly beneficial in connection with temperature sensitive materials. For example, shape, temporal width and energy amount of the single laser pulses can be appropriate for ablating any type of tissue such as bone tissue. Even though FLs might have some disadvantages compared to other pumping light sources such as laser diodes in certain cases, when the single pumping light sources are operated with comparably low pulse repetition rates such as it can be implemented for thermally sensitive materials, the lamp-specific advantages of the FLs typically dominate.

Preferably, the laser source comprises at least one laser diode as light source for the first beam generating configuration, the second beam generating configuration and/or of the third beam generating configuration. Such laser diode pumped laser can be an alternative for the flash lamps described above which can be beneficial in some applications.

In an efficient embodiment, the third beam generating configuration comprises components of the first beam generating configuration or of the second beam generating configuration. For example, the third beam generating configuration can comprise the same gain medium as any of the first or second beam generating configuration.

In another aspect, the invention is a laser device. In particular, the laser device comprises a laser source as described above, and a control unit configured to adjust the beam directing optics. Such a laser device allows for efficiently implementing and achieving the processes, effects and benefits described above in connection with the laser source according to the invention and its preferred embodiments.

In a preferred embodiment, the laser device further comprises a plume analysing arrangement adapted to identify a tissue type in a debris of a plume generated by the analysis laser beam hitting a target tissue.

The term “plume” as used herein can relate to a product of a combustion or carbonization process induced by the laser ablation and can comprise odorous molecules, smoke, aerosols and the like referred to as debris. More specifically, in the context of laser ablation, plume can summarize or comprise any substance ejected by a laser beam when hitting the target tissue as debris. Consequently, in connection with plume, the term “debris” can relate to any molecules resulting from the ablation of the target tissue such as volatile small solid fractions of the target tissue, smoke, aerosols, odorous molecules and the like.

The term “substance” as used herein can relate to a single substance, a mixture of plural substances or a pattern of a given number of masses or molecules, any spectroscopic pattern or the like.

In modern surgeries there is an increasing need to analyse tissue “online” during the intervention so that the surgeons have all possible information available during the operation to reduce operation time and, most likely, a second intervention. For example, in a vasectomy of a tumour the information of the tissue during the intervention is needed to distinguish between healthy and cancerous tissue. For example, in tumour recognition, precise tumour margin detection represents a central challenge during a surgical intervention such as in a tumour removal in bone. In such cases the surgeon needs to know if the tissue being cut surrounding the tumour is healthy or if it has also cancerous cells. For this task, analysis usually done by a number of biopsies is too slow and the surgeon opt to cut additional tissue in order to enhance certainty that the carcinogen tissue is removed. Indeed, and despite the recent technological advances, biopsies remain time consuming and rather cumbersome procedures. Furthermore, standard biopsies are sometimes conducted post-operatively depending on the results of the biopsy it might be necessary to proceed to a subsequent surgical intervention. In other words, such processes do not allow for reacting during the intervention depending on the outcome of the biopsy as desired by surgeons and patients. Therefore, online analysis during cutting can give the needed information to cut only tumour tissue and to shorten the surgery time.

Similarly, the plume analysing arrangement of the laser device allows for providing a quick and reliable identification of the target tissue such that the optimal ablation laser beam can be selected. In particular, by implementing the plume analysing arrangement into the laser device, an operation independent from any external analysis means or the like can be provided.

The principle of operation behind such laser device is that the analysis laser beam creates a plume or micro-plasma with debris from the targeted tissue. This plume counter-propagates approximately along the direction of the incoming ablating laser beam after every laser pulse strikes the target tissue. Such debris comprises molecules, atoms, fragments of cells as well as ions and electrons in the form of debris. The composition of the debris is indicative of the tissue being ablated. Thus, it can be a characteristic, or a “signature”, of the ablated tissue type.

Thereby, the control unit preferably is configured to automatically activate either the first beam generating configuration of the laser source or the second beam generating configuration of the laser source dependent on the tissue type identified by the plume analysing arrangement. More specifically, the plume analysing arrangement preferably is adapted to identify a hydrophilic tissue type and a hydrophobic tissue type.

Like this, the laser device allows accurately cutting of hydrophilic and hydrophobic tissues and analysing the tissue during a medical and particularly surgical intervention in a comparably fast and precise manner and, advantageously, within the time of the surgical intervention. The proposed laser device may eliminate the need of post-operative time-consuming biopsy by an optical biopsy and, thus, to possibly avoid a second intervention.

The plume analyzing arrangement can comprise a laser spectroscope. The laser spectroscope can comprises a laser induced fluorescence (LIF) spectroscope, a coherent anti-Stokes Raman scattering spectroscope (CARS), a laser photo-acoustic spectroscope (LPAS), a laser induced breakdown spectroscope (LIBS), an atomic emission spectroscope (AES), a AES/LIBS, a resonance-enhanced multi-photon ionization (REMPI) spectroscope, a mass spectroscope (MS), a system where molecules are separated by their collision cross section such as a ion mobility spectroscope (IMS), or an elastic scattering (ES) spectroscope. The choice of a particular laser spectroscope can depend on the specific problem at hand. Also, in some applications it might be advantageous to combine plural of these laser spectroscopes in one single laser device. For example, a combination of optical coherence tomography (OCT), LIBS and mass spectrometry (MS) can be particularly beneficial.

The laser spectroscope allows for precisely identifying and quantifying substances in the debris of the plume. Such spectroscope also allows for probing the originated plume in real-time with a specific laser beam. Thus, it allows for a comparably quick analysis such that the substances can be identified more or less in real-time time or, at least, within the time of the intervention. The term “real-time” in this connection can relate to an operation of the laser device in which the pulsed ablating laser beam is provided without any restrictions and the plume evaluation is performed during operation. An essential delay and particularly a break in the operation of the laser device is prevented.

The laser device having a laser spectroscope can also be used to determine or analyze the tissue remaining in the surface of the just ablated region instead of the ejected debris in the plume. In specific embodiments, it would even be possible that the plume analyzing arrangement is only capable of analyzing the remaining tissue and that substances in the debris of the plume generated by the laser beam ablating the target tissue are not or not properly identified.

Preferably, the control unit is configured to activate the first beam generating configuration of the laser source when the tissue type identified by the plume analysing arrangement is a hydrophilic tissue type and to activate the second beam generating configuration of the laser source when the tissue type identified by the plume analysing arrangement is a hydrophobic tissue type. Thereby, the control unit preferably is configured to simultaneously activate the first beam generating configuration and the second beam generating configuration when the tissue type identified by the plume analysing arrangement is a hydrophilic tissue type or a hydrophobic tissue type.

Preferably, the control unit is configured to activate the third beam generating configuration of the laser source to ablate the target tissue to generate the debris with the plume. Like this the analysis of the target tissue can be particularly beneficial.

Preferably, the laser device further comprises a cooling system configured to cool a target tissue hit by the primary ablating laser beam or by the secondary ablating laser beam.

Preferably, the control unit is configured to synchronize pulses of the primary ablating laser beam, the secondary ablating laser beam and the analysis laser beam.

The control unit can additionally be configured to perform various other tasks. In particular, the control unit can be a central control unit controlling the complete laser device or most portions thereof. The control unit can comprise a computer or a processing unit, a data storage, a memory and the like.

In still another aspect, the invention is a method of cutting a tissue by means of a laser device as described above. The method comprises the steps of: (i) positioning a tissue in an area of operation of the laser device where the directing optics of the laser source direct the laser beams of the laser source; (ii) the laser source of the laser device propagating an analysis laser beam generated by the third laser beam generating configuration; (iii) identifying a major tissue type in a plume of a debris generated by the analysis laser beam hitting the tissue; (iv) selecting either the first beam generating configuration or the second beam generating configuration suiting the identified major tissue type; and (v) ablating the tissue by means of the selected first laser generating configuration or second laser generation configuration of the laser source.

Such a method allows for efficiently implementing and achieving the processes, effects and benefits described above in connection with the laser device according to the invention and its preferred embodiments.

Thereby, the steps of identifying the major tissue type and the selecting the first beam generating configuration or the second beam generating configuration preferably are automatically executed by a plume analysis arrangement of the laser device.

The method preferably comprises a step of predefining an ablation geometry, wherein the target tissue is ablated by the selected first laser generation configuration or second laser generation configuration of the laser source along the ablation geometry. Thereby, the cut geometry can be predefined by a series of adjacent target spots, wherein each of the laser pulses hits the target at a predefined target spot of the series of adjacent target spots. Each of two subsequent laser pulses can hit the target at two different target spots of the series of adjacent spots, wherein the two target spots are not adjacent to each other. Also, for the most common cases encountered cases when single ablating pulses do not suffice to cut the entire tissue in question, the process can be repeated by sweeping over the same path many times until the surgical process is completed.

Still further, the method can be an in-vitro method or, alternatively, an in-vivo method.

### BRIEF DESCRIPTION OF THE DRAWINGS

Aspects of the laser source according to the invention, the laser device according to the invention and the method according to the invention are described in more detail herein below by way of an exemplary embodiment and with reference to the attached drawings, in which:

FIG. 1 shows a schematic view of a setup of an embodiment of a laser source according to the invention in an embodiment of a laser device according to the invention suitable for performing an embodiment of a method according to the invention for the ablation of a tissue depending of its water content as well as the identification of the tissue being analysed;

FIG. 2 shows a schematic detailed view of further components of the laser device of FIG. 1;

FIG. 2a shows the timing of two ablating laser beams generated by the laser source of FIG. 1 to each other, where At is the time shift or delay between them in an alternating mode;

FIG. 2b shows the timing of the two ablating laser beams generated by the laser source of FIG. 1, when one ablating laser beam is firing after two laser shots of the other ablating laser beam;

FIG. 2c shows the timing when only an analysis laser beam generated by the laser source of FIG. 1 is firing;

FIG. 2d shows the timing of the two ablating laser beams generated by the laser source of FIG. 1, when one ablating laser beam is firing after two laser shots of the other ablating laser beam;

FIG. 3a shows a schematic view of two possible power supplies used in combination with the laser source of FIG. 1;

FIG. 3b shows a schematic view of two other possible power supplies used in combination with the laser source of FIG. 1.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

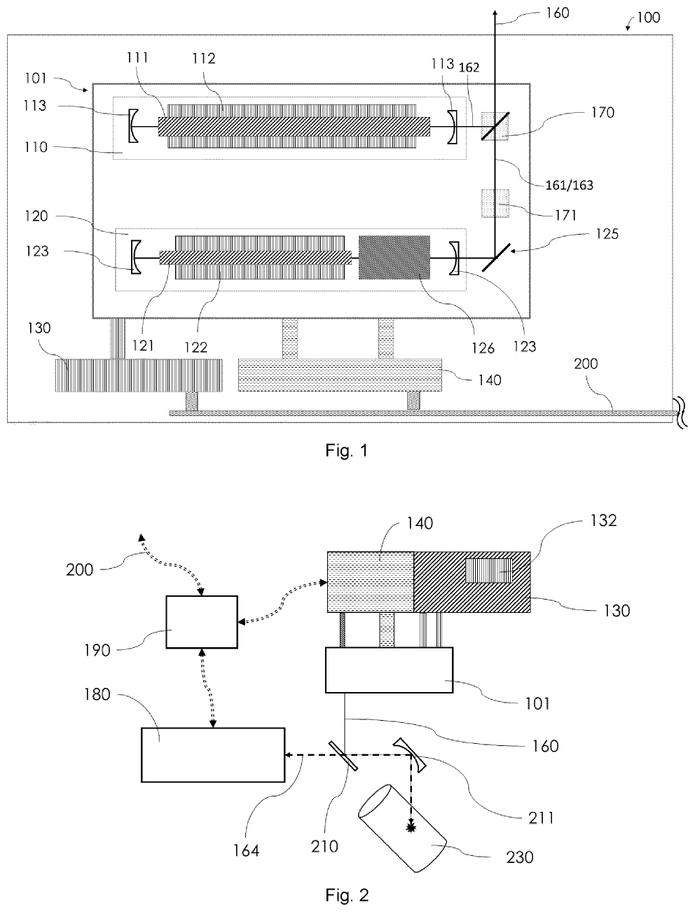

FIG. 1 shows an embodiment of a laser device 100 according to the invention equipped with an embodiment of a laser source 101 according to the invention and implementing an embodiment of a method according to the invention. The laser device 100 is in the following also referred to as universal tissue laser device 100 or UTL device 100.

The laser source 101 comprises a first flash lamp (FL) 112 arranged to pump an Er:YAG solid state rod 111 as a first gain medium, and two first resonator mirrors 113 embedding the Er:YAG solid state rod 111. The first flash lamp 112, the Er:YAG solid state rod 111 and the two first resonator mirrors 113 together form a first beam generating configuration 110, which is also referred to as first laser 110. The first beam generating configuration 110 is adapted to generate a pulsed primary ablating laser beam 162 with pulses having a first emission spectrum and a first temporal pulse width as described in more detail below.

The laser source 101 further comprises a second flash lamp (FL) 122 arranged to pump an Nd:YAG rod 121 as a second gain medium, two second resonator mirrors 123 embedding the Nd:YAG rod 121, and a Q-switching device 126 as optoelectronic element. The second flash lamp 122, the Nd:YAG solid state rod 121 and the two second resonator mirrors 123 together form a second beam generating configuration 120, which is also referred to as second laser 120. Furthermore, the same second flash lamp 122, the Nd:YAG solid state rod 121 and the two second resonator mirrors 123 form together with the Q-switching device 126 a third beam generating configuration 120, which is also referred to as second or third laser 120. The second beam generating configuration 120 is adapted to generate a pulsed secondary ablating laser beam 163 with pulses having a first emission spectrum and a first temporal pulse width as described in more detail below. The third beam generating configuration 120 is adapted to generate a pulsed analysis laser beam 161 with pulses having a third emission spectrum and a third temporal pulse width shorter than the first temporal pulse width and shorter than the second temporal pulse width as described in more detail below.

The laser source 101 is additionally equipped with a beam directing and shaping optics 125 with plural mirrors as beam aligning elements adapted to align the primary ablating laser beam 162, the secondary ablating laser beam 163 and the analysis laser beam 161 such that the laser source 101 propagates the three laser beams 160 along a same propagation path. The beam directing optics 125 further have a beam shaping optics 171 to correct for the different divergence or the primary ablating laser beam 162 and the analysis laser beam 161, and a first beam combining element 170 to combine the combined secondary ablating and analysis laser beam 161/163 with the primary ablating laser beam 162.

Besides the laser source 101 the UTL device 100 comprises a central power supply 130, a central cooling system 140 and a bus 200 for communication between the UTL device 100 and further components such as a robot to guide the laser source 101.

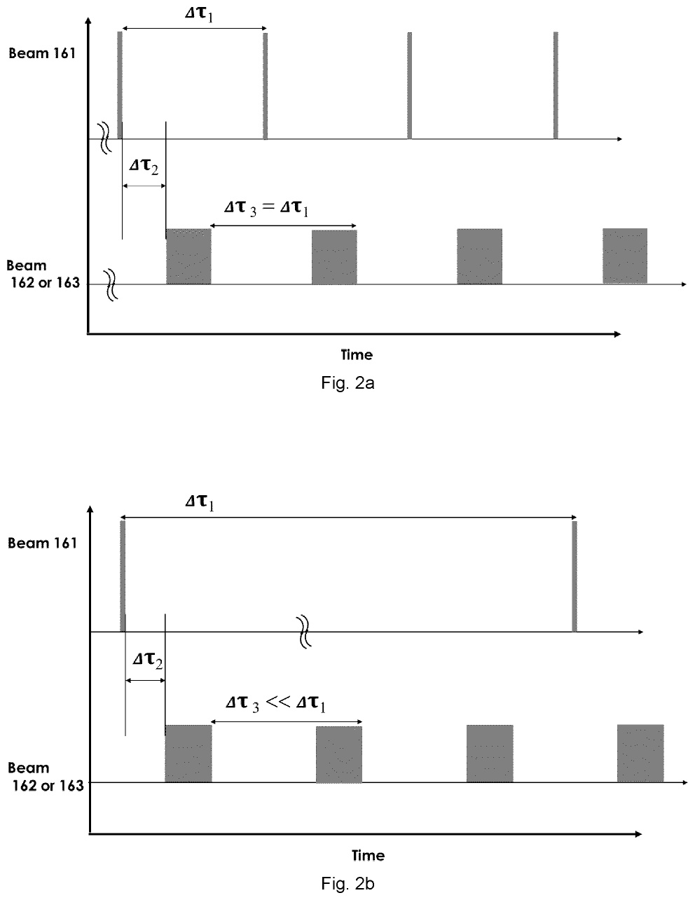

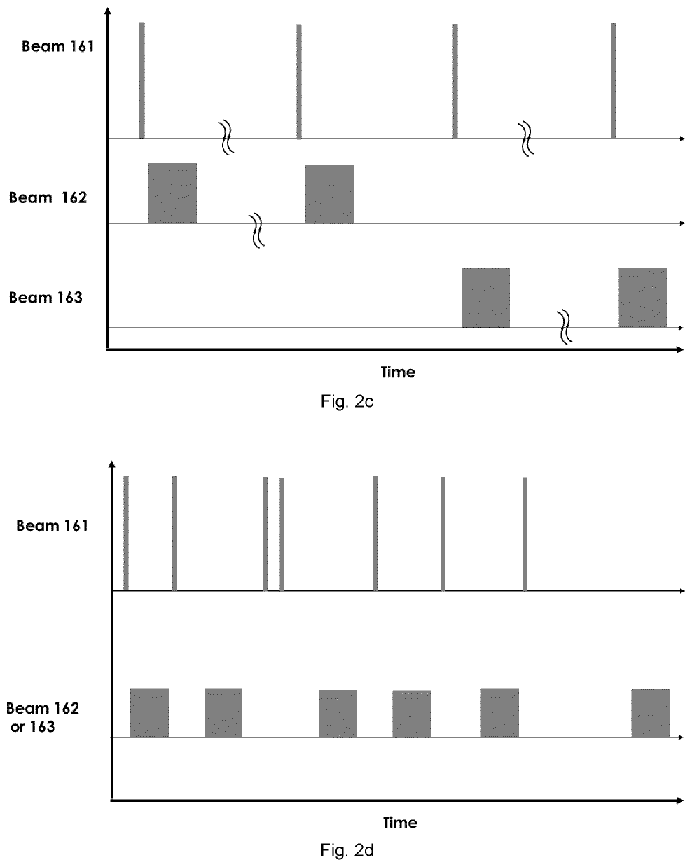

In FIG. 2 additional components of the UTL device 100 are shown. In particular, the UTL device 100 further comprises a control unit 190, an analysis unit 180 as plume analysing arrangement, a beam splitting unit 210, a beam focussing element 211 and an electronics 132 for the Q-switching device 126. The control unit 190 is coupled to the cooling system 140, the power supply 130, the analysis unit 180 and external components such as the robot to guide the laser source 101 via the bus 200. The electronics 132 is embedded in the power supply 130 which energizes the complete UTL device 100.

The beam splitting unit 210 is positioned in the propagation path. It is arranged to direct the three laser beams 160 towards the beam focussing element 211 where they are focussed and directed towards a target tissue 230. Light reflected or emitted, e.g. fluorescence from some of the fragments of the tissue converted into debris, from the target tissue due to the interaction of the analysis laser beam 161 with the target tissue 230 can be guided back contra-propagating along the optical path and captured by the analysis unit 180. This light is referred to as analytical light 164 which is used for LIBS in the analysis unit 180. The result of the real time analysis of the captured analysis light 164 by the analysis unit 180 can be further used by the control unit 190 and/or other components to further control the ablation process or other devices.

The beam splitting unit 210 can consist of multiple opto-mechanical elements such as, e.g., mirrors, dichroic mirrors or lenses to properly align different optical pathways to each other, e.g. collinear or parallel. The beam-focusing element 211 can be a lens system, reflective optics or a combination of both. Preferably, a scanner mirror as reflective optics is adapted to focus the cutting laser beam and the imaging laser beam. Thereby, the scanner mirror can be a concave mirror mounted on a movable scanning unit which can simplify alignment and controlling. Such a reflective optics design has further the advantage of smaller losses and no chromatic aberrations when using different wavelengths. In this way, a particular efficient operation of the laser ablating device is possible.

The analysis laser beam 161 has a maximum wavelength at 1'064 nm and is operated using the Q-Switching device 126 to deliver short pulses, i.e. having a temporal width of about 10 ns, of high energy. Such analysis laser beam 161 produces a high temperature plasma that excites electronically some of the degradation products in the debris which can be analysed conveniently by laser induced fluorescence (LIF) inside the analysis unit 180. For that purpose, light reflected from the analysis laser beam 161 hitting the target tissue 230 is guided to the analysis unit 180 as analytical light 164. Furthermore, the UTL device 100 also applies analysis with laser induced breakdown spectroscopy (LIBS). In particular, the analysis laser beam 161 tightly focused on the target tissue 230 generates a plume in which the debris has some of the following ions when the target tissue is a biological tissue and particularly a bone tissue: Ca⁺⁺, Mg⁺⁺, Na⁺, K⁺, H⁺, O²⁻ but also other ions. These ions have long lifetime decaying emission in the visible part of the spectrum that can be easily monitored using LIBS in the analysis unit 180. Other elements that are detectable in the debris of the plume are Fe⁺⁺⁺ and other ions. The ratios of the emission intensities of such excited elements are correlated with the type of tissue. Based on the identified type of tissue the control unit 190 selects which ablation laser beam 162, 163 to use. For LIBS surface analysis, the short laser pulses of the analysis laser beam 161 are particularly efficient to generate an element emission spectrum. However, also other laser beams with other wavelengths can be used for the same purpose provided that such laser can generate a plasma of at least 3'000 Kelvin within typically ns or even shorter pulses, because they may be less destructive for the targeted tissue.

In principle, LIBS can analyse any matter regardless of its physical state, be it solid, liquid or gaseous because all elements emit light of characteristic frequencies when excited to sufficiently high temperatures. When the components of a material to be analysed are known, LIBS may be used to evaluate the relative abundance of each constituent element, or to monitor the presence of impurities. Because comparably small amount of material is consumed during the LIBS process, the technique is considered essentially non-destructive or minimally-destructive, and with a total average power of less than one watt at the target there is almost no heating surrounding the ablation site. LIBS is also a very rapid technique giving results within seconds, making it particularly useful for the purpose at hand, i.e. real-time. LIBS is an entirely optical technique such that it requires only optical access to the specimen. And being an optical, non-invasive and non-contact techniquemakes LIBS particularly suitable and efficient to be implemented in the UTL device 100.

Technically, LIBS can be done by double laser pulses by one or different laser wavelengths, where the delay between both laser pulses is in the range of 5 μs or shorter. The first laser pulse is used to produce a clean and dry surface only while the second pulse is used for analysing the pure tissue surface. Using a low intense Er:YAG laser pulse in free-running mode to ablate biofluids and water, whereas the subsequent laser short pulse from activating the Q-switching device 126 of the third laser 120 hit the pure target tissue 230.

The UTL device 100, with its analysing laser beam 161 allows the tissue surface analysis before subsequent cutting with the ablating laser beams 162, 163. Also, during cutting by means of any of the primary or secondary ablating laser beams 162, 163, the short analytical pulses of the analysis laser beam 161 can be used at any time to generate proper debris to be conveniently analysed by the analysis 180, e.g. applying LIBS. At any time the control unit 190 may select the appropriate ablating laser beam 162, 163 in accordance with the tissue type identified by the analysis unit 180.

Among lasers that are used to ablate substrates such as human hard tissue and particularly bone tissue, solid state Erbium (Er) lasers emitting at 2'964 nm being a wavelength strongly absorbed in water are emerging as the most suitable for various technical reasons. Particularly, they can provide a high absorption of water at their 2'964 nm wavelength emission line with the possibilities to miniaturize them to be integrated into the medical device 100 and, comparably low servicing requirements. Therefore, this type of laser is embodied in the first beam generating configuration 110.

Other lasers having similar benefits used to ablate substrates such as human hard tissue and particularly bone tissue are solid state Holmium (Ho) lasers emitting light at a similar wavelength whereas these later lasers appear most suitable for internal medicine because it is easier to find waveguides to be used to bring the laser light into the body by means of, e.g., endoscopes. Er lasers are better suitable than Ho lasers for light propagation in the free-space such as air for open surgeries whereas Ho lasers for e.g. minimally invasive surgical interventions because light can be launched into optical fibres of any type.

Er:YAG and Nd:YAG crystals, where YAG stands for Yttrium Aluminium garnet (YAG=Y3Al5O12), as employed in the first gain medium 111 and the second gain medium 121, respectively, are pumped with the first and second flash lamps 112, 122 but could alternatively also be pumped with laser diodes (LDs). They are often used in flash lamp pumped Q-switched lasers to shorten pulse durations. In the context of the present invention the Nd:YAG laser will be used in both, a) the so-called free-running mode to produce relatively long pulses in the microsecond range depending primarily on the time width of the pumping FL, as embodied by the second beam generating configuration, as well as in b) the Q-switched mode to generate short pulses in the nanosecond range, as embodied by the third laser beam generating configuration. The Er:YAG laser, as embodied by the first beam generating configuration, will be used exclusively in the free-running mode delivering pulses of more than 100 microseconds.

LD pumped LD-Er:YAG and Nd:YAG lasers can be more efficient in transferring energy to create population inversion than when pumped by FLs (i.e. FL-Er:YAG) and they are easier to miniaturize with regard to their optics as well as to their electronics. Also, LD pumped Er and Nd lasers can be operated at higher repetition rates such as up to kHz repetition rates than FL pumped lasers which are usually operated at 10 to 20 Hz. Both lasers can be operated in free-running or Q-switched mode. In the context of the present invention FL pumped lasers are used.

The FL 112, 122 are used for high pulsed energies. They are rather inefficient because they produce a broad spectrum of light causing most of the energy to be wasted as heat in the gain medium whereas DL have a sharp wavelength emission and thus less energy is lost in the form of heat.

In summary, advantages of FL-Er:YAG lasers are: comparably high pump power (particularly peak power) can be generated; the price per watt of generated pump power is comparably low; and the lamps are fairly robust, e.g. immune to voltage or current spikes. Their disadvantages are: the lifetime is comparably limited (usually some hundred or up to a few thousand operating hours or, in terms of flashes about five million shots); the electrical energy to light conversion efficiency of the laser is comparably low (typically at most a few percent); and the electric power supplies usually involve high voltages which raise additional safety issues when it comes to a medical device. Consequences of the low conversion efficiency are not only higher electricity consumption but also a higher heat load, which can make necessary a more powerful cooling system.

Disadvantages of LD-Er:YAG as compared to FL-Er:YAG particularly in the context of human or animal tissue ablation purposes are the poorer quality of the laser beam (i.e. higher M2) which makes focusing comparably difficult and the comparably low peak power in long pulses degrading the ratio of electromagnetic energy that is transformed into debris as compared with that is converted reaching heat in the remaining tissue walls of, e.g., a bone being cut.

Advantages of free-running FL-Er:YAG as compared to LD-Er:YAG particularly within certain limits, can be that the former lasers can be controlled with a long time window among relatively shorter pulses such as less than 400 μs which allows to enhance the ratio of electromagnetic energy that is transformed into debris with respect to h eat flowing into the walls as compared to the LD-Er:YAG having e.g. the same total energy (e.g. 10 W in pulses having time widths of e.g. 1 ms or even longer) in low peak powers due to the much longer pulse widths and a big fraction of its energy flows as compared to heat in the remaining tissue walls of, e.g., when a bone tissue being cut.

The cooling of the lasers is embodied by the single cooling system 140 considering that in most cases only one of the first and second lasers 110, 120 is active at the same time. The tubing of the cooling is thus connected in line, i.e. the cooling liquid pass through one laser 110, 120 first and then through the other laser 110, 120.

As mentioned, the UTL device 100 can be mounted on a robotic device or any other actuating device for positioning as part of a medical device or any device that communicates to the UTL device 100 via the bus 200. Thereby, the UTL device 100 can be configured as the “slave” and the medical device as “master”.

FIG. 2a to FIG. 2d show schematic illustrations of various modes of operation with possible firing sequences. In FIG. 2a there is a pulse of the analysing laser beam 161 to generate plume with a small amount of debris to determine what kind of tissue is encountered using the analysis unit 180 embodying LIBS. Depending on this information single pulses of either one of the primary and secondary ablating laser beams 162, 163 are fired. The time space between the pulses, Δτ, of the analysis laser beam 161 and those of either pulses of the ablating laser beams 162, 163 are of identical repetition rate, i.e. frequency Δτ(1).

However, and considering that the amount of tissue to be encountered does not change over many ablation laser beam 162, 163 pulses, a user could choose to fire analysis laser beam 161 at much lower repetition rate spaced by a longer time Δτ(2) as shown in FIG. 2b. In such case the repetition rate of analysis laser beam 161, Δτ(2) could be conveniently chosen, but not necessarily, to be an even fraction of that of ablating laser beams 162, 163, Δτ(1).

FIG. 2c shows a case similar to the one shown in FIG. 2b displaying the transition from ablating with the primary ablating laser beam 162 to the secondary ablating laser beam 163.

FIG. 2d correspond to a firing arrangement where the repetition rates of any of the analysis and ablating laser beams 161, 162, 163 are not constant. Such situation could be encountered when the analysis unit 180 requires more time to determine the composition of the debris in the plume and thus what type of tissue is being ablated therefore each pulse will have a different time spacing Δτ.

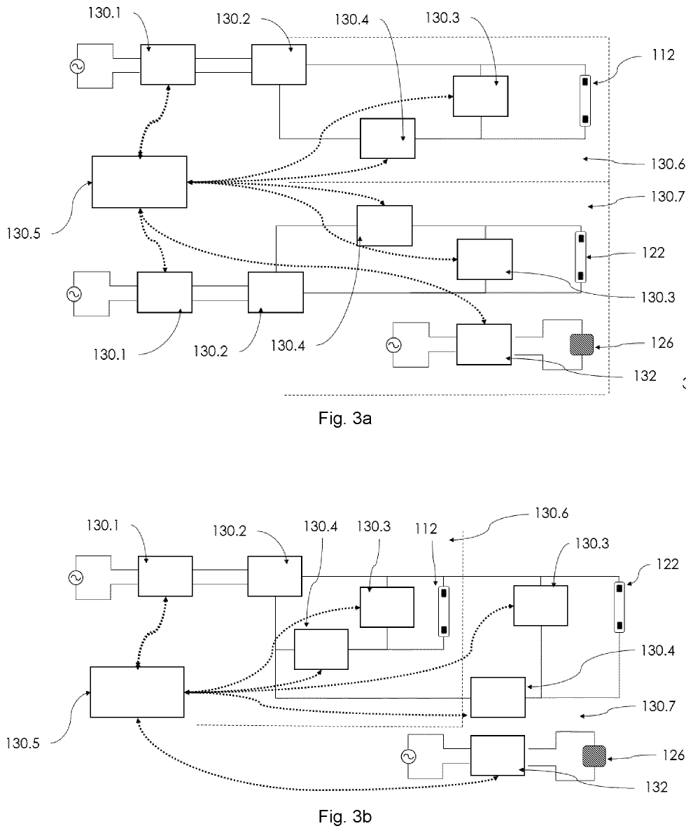

FIG. 3a depicts a simplified schematically overview of the power supply 130 consisting of two separate power circuits. One circuit 130.6 is for the primary or first laser 110 and another circuit 130.7 for the secondary or second laser 120. An additional third circuit 132 is used to control the Q-Switching device 126. All three circuits are controlled by a power supply controller 130.5 which can also control the cooling system 140 and is connected to the control unit 190 which defines the pulse setting and flashing modes.

Each power circuit 130.6, 130.7 is arranged to fire either the first laser 110 or the second laser 120. In the power circuits 130.6, 130.7 there is a charging circuit 130.1 responsible to transform the AC, i.e. alternate current supply, input to a defined DC, i.e. direct current, voltage. A capacitor unit 130.2 stores the required energy and is charged by the charging circuit 130.1 to the defined voltage level. The combination of the capacitor and charging circuit is designed such that sufficient energy is present for all applicable pulse shapes and repetition rates required in the FLs. In parallel to the FL 112, 122 there is an ignition circuit 130.3 which ignites the lamp by means of a high voltage in the range of kilo-volts applied to the FL 112, 122. To keep the respective FL 112, 122 ignited after the ignition, a simmer circuit within the pulse circuit 130.4 applies a DC-voltage to the lamp. The controller 130.5 closes the circuit over the FL 112, 122 by means of a switch integrated within the pulse circuit 130.4 for the defined time of the pulse width. This leads to flashing of the FL 112, 122 for a desired pulse width. Such a switch can be realized with any high power switch.

The power supply circuit 132 for the Q-Switching device 126 depends upon the used Q-Switching device 126. If, e.g., an electro-optic device is used the power supply 132 has to provide high voltages in the range up to kilovolts. If, e.g., an acousto-optic device is used the Q-Switching power supply 132 can embody a high RF-circuit supplying frequencies in the range of hundreds of megahertz.

FIG. 3b shows, compared to FIG. 3a, a special combination of the two power circuits 130.6 for the first laser 110 and the power circuit 130.7 of the second laser 120. In this embodiment there exists only one charging 130.1 and one capacitor circuit 130.2 for both power circuits. This simplifies the design, however, restricts flexibility of pulsing the first and second lasers 110, 120 at any time independently.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below. For example, whereas most examples and explanation above are in the field of surgery, the principle underlying the present invention also be used in other technical fields. In particular, the invention can be useful for cutting any heterogeneous substrate which advantageously is cut with different wavelengths and/or pulse widths. Or, it is possible to operate the invention in an embodiment having more than three beam generating configurations for providing additional ablating and/or analysis laser beams.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1.-28. (canceled)

29. A laser source comprising:
a first beam generating configuration adapted to generate a pulsed primary ablating laser beam with pulses having a first emission spectrum and a first temporal pulse width;
a second beam generating configuration adapted to generate a pulsed secondary ablating laser beam with pulses having a second emission spectrum different from the first emission spectrum and a second temporal pulse width;
a third beam generating configuration adapted to generate a pulsed analysis laser beam with at least one pulse having a third emission spectrum and a third temporal pulse width shorter than each of the first temporal pulse width and the second temporal pulse width; and
a beam directing optics with beam aligning elements adapted to align the primary ablating laser beam, the secondary ablating laser beam and the analysis laser beam such that the laser source propagates the laser beams along a same propagation path.

30. The laser source of claim 29, wherein the first beam generating configuration has a first gain medium to generate the primary ablating laser beam, and the second beam generating configuration has a second gain medium different from the first gain medium to generate the secondary ablating laser beam, and
wherein the third beam generating configuration comprises the second gain medium.

31. The laser source of claim 29, wherein the third beam generating configuration comprises a giant pulse former.

32. The laser source of claim 31, wherein the giant pulse former has an optoelectronic element, such as a Q-switching device, or
wherein the third beam generating configuration comprises two resonator mirrors and the giant pulse former has a rotator to which one of the two resonator mirrors of the third beam generating configuration is mounted.

33. The laser source of claim 29, wherein the first emission spectrum has a maximum in a range of about 2,900 nm to about 3,000 nm, in a range of about 2,950 nm to about 2,980 nm or in a range of about 2,960 nm to about 2,970 nm, or of about 2,964 nm.

34. The laser source of claim 29, wherein the second emission spectrum has a maximum in a range of about 1'000 nm to about 1'100 nm, in a range of about 1'050 nm to about 1'080 nm or in a range of about 1'060 nm to about 1'070 nm, or of about 1'064 nm.

35. The laser source of claim 29, wherein the third emission spectrum has a maximum in a range of about 500 nm to about 560 nm, or in a range of about 520 nm to about 540 nm, or of about 532 nm.

36. The laser source of claim 29, wherein the beam directing optics comprises a beam combining element arranged to combine the primary ablating laser beam, the secondary ablating laser beam and the analysis laser beam.

37. The laser source of claim 29, wherein the first temporal pulse width and the second temporal pulse width are in a range of about 1 μs to about 1 ms or in a range of about 150 μs to about 300 μs.

38. The laser source of claim 29, wherein the third temporal pulse width is in a range of about 1 ps to about 100 ns or in a range of about 1 ns to about 50 ns.

39. The laser source of claim 29, comprising:
at least one flash lamp as a light source of the first beam generating configuration, the second beam generating configuration and/or of the third beam generating configuration, and/or
at least one laser diode as a light source of the first beam generating configuration, the second beam generating configuration and/or of the third beam generating configuration.

40. A laser device comprising:
the laser source according to claim 29; and
a control unit configured to adjust the beam directing optics.

41. The laser device of claim 40, further comprising a plume analysing arrangement adapted to identify a tissue type in a debris of a plume generated by the analysis laser beam hitting a target tissue.

42. The laser device of claim 41, wherein the control unit is configured to automatically activate either the first beam generating configuration of the laser source or the second beam generating configuration of the laser source dependent on the tissue type identified by the plume analysing arrangement, and
wherein the plume analysing arrangement is adapted to identify a hydrophilic tissue type and a hydrophobic tissue type.

43. The laser device of claim 42, wherein the control unit is configured to activate the first beam generating configuration of the laser source when the tissue type identified by the plume analysing arrangement is a hydrophilic tissue type and to activate the second beam generating configuration of the laser source when the tissue type identified by the plume analysing arrangement is a hydrophobic tissue type, and
wherein the control unit is configured to simultaneously activate the first beam generating configuration and the second beam generating configuration when the tissue type identified by the plume analysing arrangement is a hydrophilic tissue type or a hydrophobic tissue type.

44. The laser device of claim 41, wherein the control unit is configured to activate the third beam generating configuration of the laser source to ablate the target tissue to generate the debris with the plume, and/or
to synchronize pulses of the primary ablating laser beam, the secondary ablating laser beam and the analysis laser beam.

45. The laser device of claim 40, further comprising a cooling system configured to cool a target tissue hit by the primary ablating laser beam or by the secondary ablating laser beam.

46. The laser device of claim 40, wherein the third beam generating configuration comprises components of the first beam generating configuration or of the second beam generating configuration.

47. A method of cutting a tissue by means of a laser device according to claim 40, comprising:
positioning a tissue in an area of operation of the laser device where the beam directing optics of the laser source direct the laser beams of the laser source;
the laser source of the laser device propagating an analysis laser beam generated by the third laser beam generating configuration;
identifying a major tissue type in a plume of a debris generated by the analysis laser beam hitting the tissue;
selecting either the first beam generating configuration or the second beam generating configuration suiting the identified major tissue type; and
ablating the tissue by means of the selected first laser generation configuration or second laser generation configuration of the laser source.

48. The method of claim 47, wherein the steps of identifying the major tissue type and of selecting the first beam generating configuration or the second beam generating configuration are automatically executed by a plume analysis arrangement of the laser device.

49. The method of claim 47, comprising a step of predefining an ablation geometry, wherein the target tissue is ablated by the selected first laser generation configuration or second laser generation configuration of the laser source along the ablation geometry.
